(19) United States
(12) Patent Application Publication
(10) Pub. No.: US 2026/0260721 A1
(43) Pub. Date: Sep. 3, 2026

(54) INTEGRATION OF IMAGING RADIOMICS AND PROTEOMICS FOR CHARACTERIZATION OF MYOCARDIAL FIBROSIS

(71) Applicants: Case Western Reserve University, Cleveland, OH (US); University Hospitals Cleveland Medical Center, Cleveland, OH (US); The United States Government as Represented by The Department of Veteran Affairs, Washington, DC (US)

(72) Inventors: Gargi Vijayan Pillai, Cleveland, OH (US); Satish E. Viswanath, Beachwood, OH (US); Mirela Dobre, Orange, OH (US)

(21) Appl. No.: 19/541,564

(22) Filed: Feb. 17, 2026

Related U.S. Application Data
(60) Provisional application No. 63/764,882, filed on Feb. 28, 2025.

(51) Int. Cl. G16H 10/60; A61B 5/055; G06T 7/00

(52) U.S. Cl. CPC G16H 10/60; A61B 5/055; G06T 7/0012; G06T 7/0016; G06T 2207/30061

(57) ABSTRACT

Systems, methods, and circuitries are provided for generating a quantification of a progression of myocardial fibrosis for a patient. In one example, an apparatus includes a memory device configured to store image data representing a myocardial wall of a heart of a patient, analysis circuitry, and signature model circuitry. The analysis circuitry is configured to extract a plurality of radiomic features from the image data. The signature model circuitry is configured to process the radiomic features using a signature model to generate a quantification of a progression of myocardial fibrosis for the patient.

### CROSS REFERENCE TO RELATED APPLICATIONS

This application claims the benefit of priority from U.S. Provisional Patent Application Ser. No. 63/764,882 filed on Feb. 28, 2025, entitled INTEGRATION OF IMAGING RADIOMICS AND PROTEOMICS FOR CHARACTERIZATION OF MYOCARDIAL FIBROSIS, the contents of which are hereby incorporated by reference in their entirety for all purposes.

### FEDERAL FUNDING INFORMATION

This invention was made with government support under CA280981 and HL165218 awarded by the National Institutes of Health; W81XWH-21-1-0725 awarded by the Department of Defense; 2320952 awarded by the National Science Foundation; and 1I01BX006439-01 awarded by the Department of Veterans Affairs. The government has certain rights in the invention.

### BACKGROUND

Cardiovascular disease (CVD) is a leading cause of morbidity and mortality for patients with chronic kidney disease (CKD). Myocardial fibrosis is a common occurrence in end-stage kidney disease (ESKD) and may be a strong independent predictor of major adverse cardiovascular events.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates some embodiments of an apparatus comprising a machine learning component configured to generate a signature model that generates a quantification of a progression of myocardial fibrosis for a patient based on a combination of radiomic features and plasma proteomic data, in accordance with various aspects described.

FIG. 2 illustrates an example experimental workflow used to generate the signature model of FIG. 1, in accordance with various aspects described.

FIG. 3 illustrates some embodiments of an apparatus configured to generate a quantification of a progression of myocardial fibrosis for a patient by evaluating the signature model of FIG. 1, in accordance with various aspects described.

FIG. 4 is a flow diagram outlining an example method for generating a quantification of a progression of myocardial fibrosis for a patient based on radiomic data and, optionally, proteomic data of the patient, in accordance with various aspects described.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Cardiovascular disease (CVD) is a leading cause of morbidity and mortality in patients with chronic kidney disease (CKD), accounting for over 1.2 million deaths worldwide. The risk of CVD has been shown to be higher in patients with severe CKD with up to 50% of patients with end-stage kidney disease (ESKD) suffering sudden cardiac death (SCD) due to ventricular arrhythmias. Further, the prevalence of SCD in patients exhibiting heart failure with preserved ejection fraction (HFpEF) is particularly high among CKD patients. Myocardial fibrosis, an established sequelae of uremic cardiomyopathy (i.e., the clinical phenotype of cardiac disease that accompanies ESKD), is integral to HFpEF pathogenesis and is a substrate for SCD. Early and accurate identification of CKD/ESKD patients vulnerable to developing myocardial fibrosis and SCD is critical for targeting interventions to minimize the risk of mortality.

Traditional CVD risk factors are poor predictors of uremic cardiomyopathy. There is recent evidence that cardiovascular death rates decrease after transplantation, likely because the restoration of renal function post-kidney transplant results in an improvement of cardiac function as well as a reduction in myocardial fibrosis. Concomitant improvements in ejection fraction (EF) in CKD patients with reduced myocardial fibrosis suggests that EF could be used as a surrogate when developing new predictors of severe uremic cardiomyopathy in patients with CKD, ideally by incorporating both biological as well as structural disease phenotypes.

In terms of biological phenotypes, studies have identified proteomics markers that are associated with cardiomyopathy risk in CKD patients as well as specific molecular pathways associated with HFpEF in patients with uremic cardiomyopathy. Inflammatory cytokines and chemokines have been shown to predict severity of HFpEF, albeit not specifically for uremic cardiomyopathy.

Structural assessment of CVD for CKD patients broadly relies on cardiac magnetic resonance (CMR) T1 mapping with visual examination to detect cardiac anomalies. Most previous studies have reported on differences in T1 values as a result of myocardial fibrosis, though not specifically to characterize HFpEF. However, manual identification of relevant locations on T1 maps is labor-intensive and susceptible to inter-reader variability. These issues could be addressed via radiomics, or the analysis of computer-extracted image features from radiographic imaging, toward more detailed correlation and interrogation of the myocardial wall on CMR T1 maps. While radiomic approaches have been successfully applied to diagnosis and risk stratification of multiple cardiac conditions, as well as for characterizing kidney function and renal fibrosis on CMR T1 maps, there have been no significant efforts thus far in applying radiomics for structural characterization of HFpEF on CMR T1 maps in ESKD patients.

Accordingly, the present disclosure relates an apparatus and/or method that utilizes a plurality of radiomic features, which have been extracted from digital images (e.g., cardiovascular magnetic resonance (CMR) images) of a patient with KD to generate a quantification of a progression of myocardial fibrosis for the patient. The method may access image data of a patient stored in a memory device. In some embodiments, the image data includes baseline image data and follow-up image data of the patient. A plurality of radiomic features are extracted from both the baseline image data and the follow-up image data. The plurality of radiomic features are extracted from a myocardial wall of a heart. The plurality of radiomic features may be extracted based on plasma proteomic data of the patient. The plurality of radiomic features and, optionally, the plasma proteomic data may be transferred to a machine learning model configured to generate a signature model that generates a characterization of a progression of myocardial fibrosis for a patient based on the plurality of radiomic features and, optionally, the plasma proteomic data.

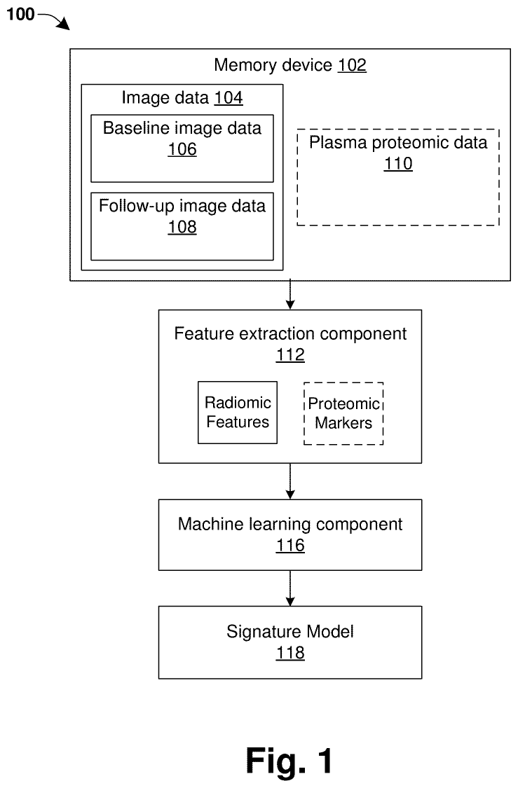

FIG. 1 illustrates some embodiments of an apparatus 100 comprising a machine learning component configured to generate a signature model 118 based on a plurality of radiomic features and, optionally, plasma proteomic data.

The apparatus 100 comprises a memory device 102 configured to store image data 104 and, optionally, plasma proteomic data 110. The image data 104 and the plasma proteomic data 110 may be of a patient that has kidney disease (KD). In some embodiments, the image data 104 comprises one or more magnetic resonance (MR) images, one or more cardiovascular magnetic resonance (CMR) images, or the like. The image data 104 may include a myocardial wall of a heart. In various embodiments, the image data 104 may comprise baseline image data 106 (e.g., an image taken of a patient at a first time) and/or follow-up image data 108 (e.g., an image taken of a patient at a second time after the baseline image data 106). The baseline image data 106 and the follow-up image data 108 may be of a same patient.

In various embodiments, the base line image data 106 is taken at a first time and the follow-up image data 108 is taken at a second time some duration after the first time, where the duration may be 1 week or greater, 1 month or greater, within a range of 1 to 12 months, or the like. In various embodiments, the baseline image data 106 and/or the follow-up image data 108 may respectively comprise one or more segmented digitized images that identify one or more regions of interest (ROI) that may, for example, be of a myocardial wall of a heart or some other suitable region. In further embodiments, the baseline image data 106 and/or the follow-up image data 108 may include annotated digitized images of the one or more ROI. The memory device 102 may, for example, comprise electronic memory (e.g., solid state memory, static random-access memory (SRAM), dynamic random-access memory (DRAM), and/or the like).

In some embodiments, the plasma proteomic data 110 comprises plasma sample data, proteomic MCSF (macrophage colony stimulating factor) data, or the like. In various embodiments, the plasma proteomic data 110 may be of the same patient as the baseline image data 106 and the follow-up image data 108. The plasma proteomic data 110 may, for example, include data and/or profiles related to plasma proteins comprising inflammatory and oxidative stress-related proteins such as Tetranectin, Myb/SANT-like DNA-binding domain-containing protein 2, Alpha-1-microglobulin, Pantothenate kinase 1, and NT-proBNP, and/or the like. The plasma proteomic data 110 may comprise baseline proteomic data and/or follow-up proteomic data. In some embodiments, the baseline proteomic data may be taken when the baseline image data 106 is taken (e.g., on or near a same day as the baseline image data 106) and the follow-up proteomic data may be taken when the follow-up image data 108 is taken (e.g., on or near a same day as the follow-up image data 108). In yet further embodiments, the plasma proteomic data 110 comprises proteomic markers and/or MCSF values associated with biological drivers of fibrosis, where the proteomic markers and/or MCSF values are generated from plasma proteomic analysis performed on plasma sample data.

A feature extraction component 112 is configured to extract a plurality of radiomic features from the image data 104. In some embodiments, the plurality of radiomic features are extracted from both the baseline image data 106 and the follow-up image data 108. In such embodiments, the plurality of radiomic features include a plurality of baseline radiomic features and a plurality of follow-up radiomic features. In various embodiments, the feature extraction component 112 is configured to extract the plurality of radiomic features from the baseline image data 106 and/or the follow-up image data 108 based on the plasma proteomic data 110. For example, proteomic markers and/or proteomic labels of the plasma proteomic data 110 may be utilized by the feature extraction component 112 in determining which radiomic features are extracted. In some embodiments, the radiomic features include first-order statistics, edge-based, co-occurrence based, wavelet-based features, and/or the like. The radiomic features may be extracted from a myocardial wall of a heart of the patient.

In yet further embodiments, the feature extraction component 112 may be configured to extract a plurality of proteomic markers from the plasma proteomic data 110. In various embodiments, the extracted plurality of proteomic markers may be utilized by the feature extraction component 112 to extract the plurality of radiomic features from the image data 104. In various embodiments, the plurality of proteomic markers are alternatively or additionally input to a machine learning component 116 along with the radiomic features for use in generating a signature model.

A machine learning component 116 is configured to generate a signature model 118 that generates medical characterization of a progression of myocardial fibrosis for the patient based on the radiomic features or based on both the radiomic features and the proteomic markers. In some embodiments, the machine learning component 116 comprises one or more machine learning models trained to generate the signature model 118 based at least in part on the radiomic features. By utilizing the plurality of radiomic features (e.g., that may be extracted based on the proteomic markers) the signature model 118 may more accurately characterize the progression of myocardial fibrosis and improve patient care. In embodiments in which the proteomic markers are input to the machine learning component 116 and used, in addition to the radiomic features (e.g., that may be extracted based on the proteomic data) the signature model 118 may more accurately characterize the progression of myocardial fibrosis and improve patient care.

Examples herein can include subject matter such as an apparatus, including a digital whole slide scanner, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system, according to embodiments and examples described.

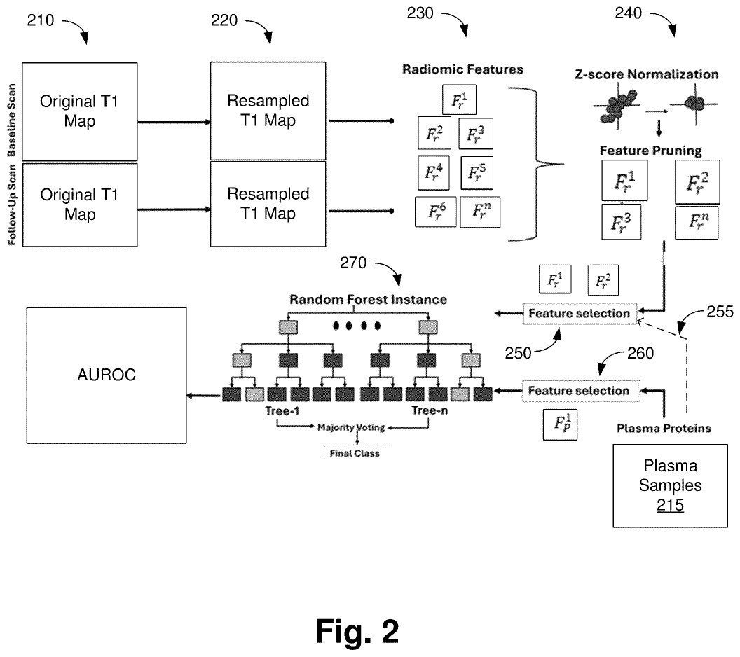

FIG. 2 illustrates an example experimental workflow that may be used to generate the signature model 118 of FIG. 1. At 210, baseline CMR T1 scans were obtained for each patient in a discovery cohort together with a separate follow-up CMR T1 scan after 9 months. Based on the distribution of extraction fraction (EF) values recorded clinically, patients were sub-grouped into those with showing less than 5% improvement in EF between follow-up and baseline time points or improved otherwise. The myocardial wall at the mid slice of all available CMR T1 maps was annotated.

Baseline plasma samples 215 were obtained from all patients in the discovery cohort for plasma proteomics analysis. Protein abundance was measured using the SomaScan v4.1 platform (SomaLogic, Inc., Boulder, CO), featuring 7,596 aptamer reagents (SOMAmers). Each SOMAmer is designed to bind a specific target protein in the sample, permitting precise quantification of multiple proteins from a small sample volume. Raw SomaScan data underwent standard preprocessing to remove outliers and normalize sample intensity distributions. Proteins targeted by multiple aptamers were aggregated at the gene level to minimize redundancy, resulting in a dataset of 6,472 proteins.

At 220, discrepancies in spatial resolution across the discovery cohort patients were identified using a MR imaging quality control package, based on which datasets were resampled to a consistent resolution of 1.41×1.41×1 mm. Two hundred and thirty radiomic features 230 were then extracted from the annotated wall region of interest (ROI) on a pixel-wise basis, from each of the baseline and follow-up scans. These included first-order statistics edge-based, co-occurrence based, and wavelet-based features; all of which have shown promise for disease characterization in previous studies. Statistical descriptors (median, variance, kurtosis, skewness) were calculated across all the pixels annotated per ROI, yielding a total of 920 radiomic descriptors associated with each patient and for each scan.

At 240, Z-score normalization was employed to ensure that extracted radiomic features had zero mean and unitary mean absolute deviation. A multi-stage combination of Spearman correlation and Maximum Redundancy Minimum Relevance (mRMR) was utilized for feature selection, to identify the most relevant features associated with improvement in EF. This was done separately for baseline and follow-up scans, resulting in two distinct ranked subsets of radiomic descriptors, denoted

$${F}_{r}^{1} and {F}_{r}^{2} ,$$

respectively. Both

$${F}_{r}^{1} and {F}_{r}^{2}$$

include descriptors quantifying edge based patterns together with a combination of intensity and gradient co-occurrence-based entropy, variance and correlation measurements from CMR T1 maps; though each feature set was different in terms of the specific statistics involved.

In some examples, as indicated by dashed line 255, the proteomic data is used in the radiomic feature selection performed at 240. In these examples a multi-stage combination of Spearman correlation and nonparametric Wilcoxon rank sum testing was utilized for feature selection. Labels defined via macrophage colony stimulating factor (MCSF) analysis were used during feature selection within the discovery cohort, to ensure that selected radiomic features were deeply correlated with underlying biological drivers of fibrosis. This was done separately for baseline and follow-up scans, resulting in two distinct ranked subsets of radiomic descriptors (based on selection frequency).

At 260 differential expression analysis with DESeq2 was conducted on 6,472 human proteins measured in the protein samples to model log-transformed protein intensities against improvement in EF. 449 proteins with a nominal p<0.05 were subsequently examined using Firth's bias-reduced logistic regression resulting in 72 proteins that remained significant with p<0.028. mRMR feature selection was then utilized to identify the most relevant proteomics markers associated with improvement in EF. A single ranked subset of proteomic markers was thus identified, as plasma samples were only obtained at baseline, denoted

$${F}_{p}^{1} .$$

A distinct profile or plasma proteins that were most closely associated with improvement in EF were selected within

$${F}_{p}^{1} ,$$

comprising inflammatory and oxidative stress-related proteins such as Tetranectin, Myb/SANT-like DNA-binding domain-containing protein 2, Alpha-1-microglobulin, Pantothenate kinase 1, and NT-proBNP.

Combined radio-proteomic signature models were developed by concatenating the individual feature subsets from baseline and follow-up, to yield

$${F}_{rp}^{1} = [ {F}_{r}^{1} , {F}_{p}^{1} ] and {F}_{rp}^{2} = [ {F}_{r}^{2} , {F}_{p}^{1} ] .$$

At 270, random forest (RF) classifiers (e.g., each classifier corresponding to a candidate signature model) were utilized to evaluate the performance of each of each feature subset or combined radio

$${F}_{r}^{1} , {F}_{p}^{1} , {F}_{r}^{2} , {F}_{rp}^{1} and {F}_{rp}^{2}$$

separately, for distinguishing between patient groupings based on improvement in EF. All feature selection and machine classification steps were repeated over 100 iterations of randomized three-fold cross-validation across the entire cohort, to ensure robustness.

Performance of RF classifiers based on

$${F}_{r}^{1} , {F}_{p}^{1} , {F}_{r}^{2} , {F}_{rp}^{1} , and {F}_{rp}^{2}$$

was evaluated via cross-validated area under the receiver-operator curve (AUROC). Based on identifying the optimal threshold in each of 100 cross-validation runs, each patient was assigned to an EF grouping (improved vs not improved). This allowed for calculation of corresponding accuracy, sensitivity, and specificity values (as well as confusion matrices) for each of the feature sets considered. Model performance was compared via non-parametric Mann-Whitney U test, with the criterion for statistical significance defined as p<0.05.

It was found that combined radio-proteomic feature subsets

$${F}_{rp}^{1}$$

### (AUROC=0.73±0.06) and

$${F}_{rp}^{2}$$

(AUROC=0.71±0.06) yielded the best overall performance when using baseline and follow-up scans, respectively. Corresponding accuracies for these predictors were 0.76 and 0.79 with only marginal differences in confusion matrices, respectively. By contrast,

$${F}_{p}^{1}$$

yielded a lower AUROC of 0.70±0.05 (p<0.05 vs

$${F}_{rp}^{1}$$

). Corresponding performance was also lower for

$${F}_{r}^{1}$$

AUROC of 0.65±0.08, p<0.05 vs

$${F}_{rp}^{1}$$

) and

$${F}_{r}^{2}$$

(AUROC of 0.62±0.08), (p<0.05 vs

$${F}_{rp}^{2}$$

). Thus either, or both, of the classifiers associated with the combined radio-proteomic feature subsets

$${F}_{rp}^{1}$$

and/or

$${F}_{rp}^{2}$$

may be selected as the signature model 118 of FIG. 1. This signature model may be used to generate a medical characterization for a particular patient based on radiomic images and proteomic data for the patient. The medical characterization may be, for example, a quantification of the progression of myocardial fibrosis in the patient.

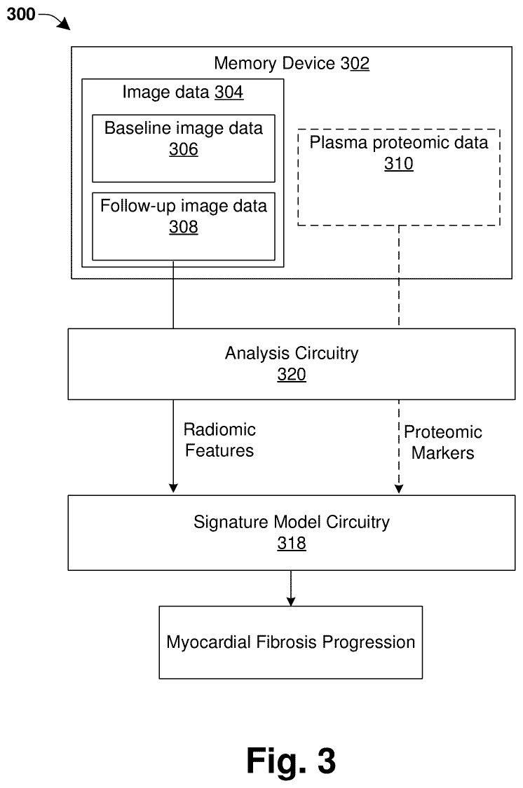

FIG. 3 illustrates an example embodiment of an apparatus 300 configured to generate a myocardial fibrosis progression for a patient based on image data and, optionally, plasma proteomic data.

The apparatus 300 comprises a memory device 302 configured to store image data 304 and plasma proteomic data 310 for a patient that has kidney disease (KD). In some embodiments, the image data 304 comprises one or more magnetic resonance (MR) images, one or more cardiovascular magnetic resonance (CMR) images, or the like. The image data 304 may include a myocardial wall of a heart. In various embodiments, the image data 304 may comprise baseline image data 306 and/or follow-up image data 308.

In various embodiments, the base line image data 306 is taken at a first time and the follow-up image data 308 is taken at a second time some duration after the first time, where the duration may be 1 week or greater, 1 month or greater, within a range of 1 to 12 months, or the like. In various embodiments, the baseline image data 306 and/or the follow-up image data 308 may respectively comprise one or more segmented digitized images that identify one or more regions of interest (ROI) that may, for example, be of a myocardial wall of a heart or some other suitable region. In further embodiments, the baseline image data 306 and/or the follow-up image data 308 may include annotated digitized images of the one or more ROI. The memory device 302 may, for example, comprise electronic memory (e.g., solid state memory, static random-access memory (SRAM), dynamic random-access memory (DRAM), and/or the like).

In some embodiments, the plasma proteomic data 310 comprises plasma sample data, proteomic MCSF (macrophage colony stimulating factor) data, or the like, for the patient. The plasma proteomic data 310 may, for example, include data and/or profiles related to plasma proteins comprising inflammatory and oxidative stress-related proteins such as Tetranectin, Myb/SANT-like DNA-binding domain-containing protein 2, Alpha-1-microglobulin, Pantothenate kinase 1, and NT-proBNP, and/or the like. The plasma proteomic data 310 may comprise baseline proteomic data and/or follow-up proteomic data. In some embodiments, the baseline proteomic data may be taken when the baseline image data 306 is taken (e.g., on or near a same day as the baseline image data 306) and the follow-up proteomic data may be taken when the follow-up image data 308 is taken (e.g., on or near a same day as the follow-up image data 308). In yet further embodiments, the plasma proteomic data 310 further comprises proteomic markers and/or MCSF values associated with biological drivers of fibrosis, where the proteomic markers and/or MCSF values are generated from plasma proteomic analysis performed on plasma sample data.

Analysis circuitry 320 is configured to extract a plurality of radiomic features from the image data 304. The plurality of radiomic features correspond to the radiomic features or subsets of features identified in the process outlined in FIGS. 1 and 2. Recall that the radiomic features selected for the signature model 118 (and extracted by analysis circuitry 320 for this particular patient) may have been selected based on proteomic data of the discovery cohort. In some embodiments, the plurality of radiomic features are extracted from both the baseline image data 306 and the follow-up image data 308. In such embodiments, the plurality of radiomic features include a plurality of baseline radiomic features and a plurality of follow-up radiomic features. In some embodiments, the radiomic features include first-order statistics, edge-based, co-occurrence based, wavelet-based features, and/or the like. The radiomic features may be extracted from the image of the myocardial wall of the patient.

In yet further embodiments, the analysis circuitry 320 may be configured to extract a plurality of proteomic markers from the plasma proteomic data 310. The proteomic markers may include inflammatory and oxidative stress-related proteins such as Tetranectin, Myb/SANT-like DNA-binding domain-containing protein 2, Alpha-1-microglobulin, Pantothenate kinase 1, and NT-proBNP. In these embodiments, the analysis circuitry 320 provides the proteomic markers to the signature model 118 along with the radiomic features for use in generating myocardial fibrosis progression value. The myocardial fibrosis progression value refers generally to any quantitative characterization of the progression of myocardial fibrosis in the patient being evaluated.

The analysis circuitry 320 may include a processor configured to execute stored instructions for extracting the radiomic features from the image data 304 and the proteomic markers from the plasma proteomic data 310. The signature model circuitry 318 may be instantiated by way of a processor executing stored instructions for processing the radiomic features and, optionally, the proteomic markers using a stored signature model. The signature model used by the signature model circuitry 318 may include, for example, a weighted sum of the radiomic features and, optionally, proteomic markers, where the weights for each radiomic feature or proteomic marker are determined by the machine learning component 116 of FIG. 1.

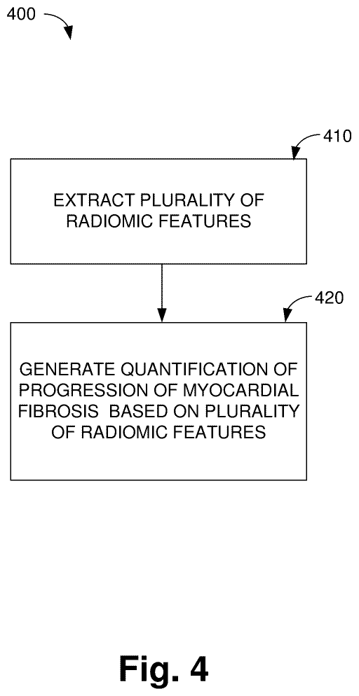

FIG. 4 is a flow diagram outlining an example method 400 for generating a quantification of a progression of myocardial fibrosis in a patient. The method may be performed, for example, by the apparatus of FIG. 3 using a signature model 118 generated as described, for example, with reference to FIGS. 1 and 2. The method includes, at 410, extracting a plurality of radiomic features from image data representing a myocardial wall of a heart of a patient. In some examples, the radiomic features are selected based on previous analysis of image data and plasma proteomic data of a cohort of discovery patients as described in FIGS. 1 and 2.

At 420, the method includes generating a quantification of a progression of myocardial fibrosis for the patient signature model circuitry based on the plurality of radiomic features. In some examples this generating of the quantification of the progression of myocardial fibrosis for the patient is performed by processing the plurality of radiomic features using a signature model (e.g., signature model 318) that generates the quantification of the progression of myocardial fibrosis for the patient based on the plurality of radiomic features. The plurality of radiomic features may include first-order statistics, edge-based, co-occurrence based, or wavelet-based features of the myocardial wall of the patient.

In some examples, the method includes extracting a proteomic marker from plasma proteomic data of the patient; and generating the quantification of the progression of myocardial fibrosis for the patient based on both the plurality of radiomic features and the proteomic marker. This may be performed by processing the plurality of radiomic features and the proteomic marker using a signature model (e.g., signature model 318) that generates the quantification of the progression of myocardial fibrosis for the patient based on the plurality of radiomic features and the proteomic marker.

In some examples, the proteomic marker includes inflammatory and oxidative stress-related proteins, for example, Tetranectin, Myb/SANT-like DNA-binding domain-containing protein 2, Alpha-1-microglobulin, Pantothenate kinase 1, or NT-proBNP.

It can be seen that the described radio-proteomic signature for characterizing HFpEF and progression of myocardial fibrosis in patients with end-stage kidney disease yields improved medical characterization of the disease in patients. By leveraging a novel multimodal feature interrogation approach, distinct sets of radiomic features from the myocardial wall on cardiac MR T1 maps and plasma proteomic markers were found to be significantly associated with improvement in ejection fraction. Integrating these complementary feature sets yielded significantly improved performance in discriminating patients with and without improvement in ejection fraction, across both baseline and follow-up time points.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. An apparatus, comprising:
a memory device configured to store image data representing a myocardial wall of a heart of a patient;
analysis circuitry configured to extract a plurality of radiomic features from the image data; and
signature model circuitry configured to process the radiomic features using a signature model to generate a quantification of a progression of myocardial fibrosis for the patient.

2. The apparatus of claim 1, wherein the plurality of radiomic features are selected based on radiomic features and plasma proteomic data of a discovery cohort of patients.

3. The apparatus of claim 1, wherein plurality of radiomic features include first-order statistics, edge-based, co-occurrence based, or wavelet-based features of the myocardial wall of the patient.

4. The apparatus of claim 1, wherein
the memory device is configured to store plasma proteomic data associated with the patient; and
the analysis circuitry is configured to extract a proteomic marker from the plasma proteomic data; and
the signature model circuitry is configured to process the plurality of radiomic features and the proteomic marker with the signature model to generate the quantification of the progression of myocardial fibrosis for the patient.

5. The apparatus of claim 4, wherein the proteomic marker comprises inflammatory and oxidative stress-related proteins.

6. The apparatus of claim 4, wherein the proteomic marker comprises Tetranectin, Myb/SANT-like DNA-binding domain-containing protein 2, Alpha-1-microglobulin, Pantothenate kinase 1, or NT-proBNP.

7. The apparatus of claim 4, wherein the plasma proteomic data comprises plasma sample data or proteomic MCSF (macrophage colony stimulating factor) data.

8. The apparatus of claim 1, wherein the quantification of the progression of myocardial fibrosis for the patient is related to a change in ejection fraction.

9. The apparatus of claim 1, wherein the image data includes a cardiovascular magnetic resonance (CMR) image T1 map.

10. A method, comprising:
extracting a plurality of radiomic features from image data representing a myocardial wall of a heart of a patient; and
generating a quantification of a progression of myocardial fibrosis for the patient based on the plurality of radiomic features.

11. The method of claim 10, comprising generating the quantification of the progression of myocardial fibrosis for the patient by processing the plurality of radiomic features using a signature model that generates the quantification of the progression of myocardial fibrosis for the patient based on the plurality of radiomic features.

12. The method of claim 10, wherein the radiomic features are selected based on radiomic features and plasma proteomic data of a discovery cohort of patients.

13. The method of claim 10, wherein the plurality of radiomic features include first-order statistics, edge-based, co-occurrence based, or wavelet-based features of the myocardial wall of the patient.

14. The method of claim 10, comprising
extracting a proteomic marker from plasma proteomic data of the patient; and
generating the quantification of the progression of myocardial fibrosis for the patient based on the plurality of radiomic features and the proteomic marker.

15. The method of claim 14, comprising generating the quantification of the progression of myocardial fibrosis for the patient by processing the plurality of radiomic features and the proteomic marker using a signature model that generates the quantification of the progression of myocardial fibrosis for the patient based on the plurality of radiomic features and the proteomic marker.

16. The method of claim 14, wherein the proteomic marker comprises Tetranectin, Myb/SANT-like DNA-binding domain-containing protein 2, Alpha-1-microglobulin, Pantothenate kinase 1, or NT-proBNP.

17. A non-transitory computer readable medium storing processor-executable instructions that, when executed by a processor, cause the processor to perform functions, comprising:
extracting a plurality of radiomic features from image data representing a myocardial wall of a heart of a patient; and
generating a quantification of a progression of myocardial fibrosis for the patient based on the plurality of radiomic features.

18. The non-transitory computer readable medium of claim 17, wherein the instructions include instructions for generating the quantification of the progression of myocardial fibrosis for the patient by processing the plurality of radiomic features using a signature model that generates the quantification of the progression of myocardial fibrosis for the patient based on the plurality of radiomic features.

19. The non-transitory computer readable medium of claim 17, wherein the radiomic features are selected based on radiomic features and plasma proteomic data of a discovery cohort of patients.

20. The non-transitory computer readable medium of claim 17, wherein the instructions include instructions for
extracting a proteomic marker from plasma proteomic data of the patient; and
generating the quantification of the progression of myocardial fibrosis for the patient based on the plurality of radiomic features and the proteomic marker.
